(19) United States
(12) Patent Application Publication
(10) Pub. No.: US 2025/0161189 A1
(43) Pub. Date: May 22, 2025

(54) COMPOSITION OF COSMETICS FOR SOOTHING AND MOISTURIZING, CONTAINING ENZYMATICALLY PROCESSED AND LOW-TEMPERATURE AGED LOW-MOLECULAR-WEIGHT HYALURONIC ACID HYDROLYSATE, AND METHOD FOR MANUFACTURING THE HYDROLYSATE

(71) Applicant: HUGEL INC., Chuncheon-si (KR)

(72) Inventors: Jin Woo MIN, Hwaseong-si (KR); Won Sang SEO, Seoul (KR)

(21) Appl. No.: 18/953,654

(22) Filed: Nov. 20, 2024

(30) Foreign Application Priority Data
Nov. 21, 2023 (KR) 10-2023-0162012

(51) Int. Cl. A61K 8/73; A61Q 19/00; C12P 19/26

(52) U.S. Cl. CPC A61K 8/735; A61Q 19/005; A61Q 19/007; C12P 19/26; A61K 2800/805

(57) ABSTRACT

Provided are a cosmetic composition for alleviating skin irritation and enhancing moisturization, including enzyme-treated and low-temperature-aged low-molecular-weight hyaluronic acid hydrolysate as an effective ingredient, and to a method of preparing the hydrolysate, wherein the low-molecular-weight hyaluronic acid prepared by the method achieves a molecular weight of 500 Da or less, resulting in significantly improved skin absorption rate and can thus provide significantly superior effects in alleviating skin irritation and providing moisturization.

### CROSS-REFERENCE TO RELATED APPLICATION

This application is based on and claims priority under 35 USC § 119 to Korean Patent Application No. 10-2023-0162012, filed on Nov. 21, 2023, in the Korean Intellectual Property Office, the disclosure of which is incorporated by reference herein in its entirety.

### BACKGROUND

### 1. Field

The present invention relates to a cosmetic composition for alleviating skin irritation and enhancing moisturization, containing enzyme-treated and low-temperature-aged low-molecular-weight hyaluronic acid hydrolysate as an effective ingredient, and to a method of preparing the hydrolysate. In particular, through processing hyaluronic acid into an enzyme-treated and low-temperature-aged hydrolysate, it is possible to obtain a low-molecular-weight hyaluronic acid hydrolysate with a weight-average molecular weight (Mw) of 500 Da or less. This hydrolysate has been confirmed to exhibit histamine inhibitory activity, nitric oxide (NO) production inhibitory activity, and moisturization-enhancing effects.

### 2. Description of the Related Art

Hyaluronic acid, as one of the glycosaminoglycans, exists in the extracellular matrix and is involved in maintaining tissue hydration and in storing and diffusing cell growth factors and nutrients, and is known to be synthesized by keratinocytes and fibroblasts. A decrease in hyaluronic acid within the skin is known to cause reduced skin elasticity and increased wrinkles.

Most hyaluronic acid produced by conventional methods is a high-molecular-weight polysaccharide that has a high degree of polymerization and has a molecular weight of 500,000 Da (500 kDa) or more, making it unable to penetrate the skin barrier. When applied topically, hyaluronic acid with a molecular weight over 500,000 Da can prevent moisture loss by attracting and binding to water molecules from the environment. However, since it cannot penetrate the skin barrier, it remains on the skin surface and is readily removed by washing, limiting its long-term moisturizing benefits. Therefore, various attempts to reduce the molecular weight of hyaluronic acid have been reported.

As prior art for reducing the molecular weight of hyaluronic acid, Patent Document 1 relates to a method for producing hyaluronic acid oligomers containing ultra-low-molecular-weight hyaluronic acid, wherein this method discloses a process for producing low-molecular-weight hyaluronic acid by inducing a deacetylation reaction. The composition is reported to have the effect of alleviating and improving wrinkles by promoting fibroblast activity and collagen synthesis.

Additionally, Patent Document 2 relates to a method of producing low-molecular-weight hyaluronic acid by gamma irradiation and hyaluronidase treatment, wherein this method provides a process for producing low-molecular-weight hyaluronic acid hydrolysate by treating hyaluronic acid with a recombinant protein enzyme and subsequently irradiating it with gamma rays. However, since this method involves the use of chemical agents or recombinant proteins that are difficult to purify, there are limitations in its industrial application.

As a result of efforts to maximize the performance of hyaluronic acid, the present inventors have discovered that when hyaluronic acid is processed into a hydrolysate by combining enzyme treatment with low-temperature aging, it is possible to achieve molecular weight reduction of hyaluronic acid while imparting excellent effects of alleviating skin irritation and enhancing moisturization, thereby completing the present invention.

### SUMMARY

Object 1 of the present invention is to provide a method of preparing low-molecular-weight hyaluronic acid.

Object 2 of the present invention is to provide low-molecular-weight hyaluronic acid having a weight-average molecular weight (Mw) of 500 Da or less, prepared by the method.

Object 3 of the present invention is to provide a cosmetic composition for alleviating skin irritation and providing moisturization, including the low-molecular-weight hyaluronic acid prepared by the method.

Object 4 of the present invention is to provide a cosmetic composition for alleviating skin irritation and providing moisturization, including low-molecular-weight hyaluronic acid having a weight-average molecular weight (Mw) of 500 Da or less.

To achieve the aforementioned objects, provided is a method of preparing low-molecular-weight hyaluronic acid, the method including: mixing hyaluronic acid and mixed enzymes including proteinase and pectinase in distilled water, and conducting an enzymatic reaction to obtain mixed-enzyme-treated hyaluronic acid (Process 1); and subjecting the mixed-enzyme-treated hyaluronic acid obtained from Process 1 to steam treatment, and subsequently to low-temperature aging treatment at about 5° C. to about 20° C. for about 1 day to about 5 days (Process 2).

In the method according to the present invention, Process 1 may be a process of hydrolyzing high weight-average molecular weight (Mw) hyaluronic acid raw material by mixed enzyme treatment. The weight-average molecular weight (Mw) of commercially available hyaluronic acid raw materials is approximately 1 million to 2 million Da.

The proteinase in Process 1 may be used alone or in combination of two or more selected from Flavourzyme, Protamex, α-chymotrypsin, subtilisin Carlsberg, thermolysin, papain, fungal protease, or pepsin.

The pectinase in Process 1 may be used alone or in combination of two or more selected from Pectinex Ultra SP-L, Viscozyme L, Ultrazyme AFP, Pectinex Ultra AFP, Pectinex Ultra Clear, or α-herbzyme.

Preferably, the mixed enzymes may include about 1 part by weight to about 5 parts by weight of proteinase with respect to 1 part by weight of pectinase, more preferably, about 2 parts by weight to about 4 parts by weight of proteinase with respect to 1 part by weight of pectinase, and even more preferably, about 3 parts by weight of proteinase with respect to 1 part by weight of pectinase.

If the weight ratio of the mixed enzymes departs from the aforementioned ranges, problems may arise due to decreased hydrolysis efficiency for hyaluronic acid, wherein the weight-average molecular weight (Mw) of hyaluronic acid may not be reduced to 500 Da or less.

In addition, in Process 1, the mixed enzymes may be used in an amount of about 2 parts by weight to about 6 parts by weight, preferably about 3 parts by weight to about 5 parts by weight, and more preferably about 3.5 parts by weight to about 4.5 parts by weight, based on 100 parts by weight of hyaluronic acid.

In the method according to the present invention, Process 2 may be a process of further reducing the molecular weight of the hydrolyzed hyaluronic acid by subjecting the same to low-temperature aging treatment.

Preferably, in Process 2, the steam treatment may be conducted at about 70° C. to about 90° C. for about 2 hours to about 4 hours, and the low-temperature aging treatment may be conducted at about 14° C. to about 18° C. for about 2 days to about 4 days.

The low-molecular-weight hyaluronic acid prepared by the method according to the present invention may be characterized by having a weight-average molecular weight (Mw) of 500 Da or less.

Provided is low-molecular-weight hyaluronic acid having a weight-average molecular weight (Mw) of 500 Da or less, prepared by the above method.

The low-molecular-weight hyaluronic acid having a weight-average molecular weight (Mw) of 500 Da or less may exhibit significantly improved skin absorption rate, thereby providing significant effects in alleviating skin irritation and providing moisturization.

Provided is a cosmetic composition for alleviating skin irritation and providing moisturization, including low-molecular-weight hyaluronic acid prepared by the above method.

Provided is a cosmetic composition for alleviating skin irritation and providing moisturization, including low-molecular-weight hyaluronic acid having a weight-average molecular weight (Mw) of 500 Da or less.

### BRIEF DESCRIPTION OF THE DRAWINGS

These and/or other aspects will become apparent and more readily appreciated from the following description of the embodiments, taken in conjunction with the accompanying drawings in which:

FIG. 1 is a graph showing results of cytotoxicity tests for hyaluronic acid samples prepared in Example 1 and Comparative Examples 1 to 4;

FIG. 2 is a graph showing results of histamine inhibition activity for hyaluronic acid samples prepared in Example 1 and Comparative Examples 1 to 4;

FIG. 3 is a graph showing results of nitric oxide (NO) production inhibitory activity for hyaluronic acid samples prepared in Example 1 and Comparative Examples 1 to 4;

FIG. 4 is a graph showing an increase rate in skin moisture content according to treatment with a hyaluronic acid sample prepared in Example 1 and a control sample (Comparative Example 1);

FIG. 5 is a spectrum image showing molecular weight measurement of the hyaluronic acid hydrolysate of Example 1; and

FIG. 6 is a table summarizing peak information obtained from a spectrum measuring molecular weight of the hyaluronic acid hydrolysate of Example 1, and the gray cells indicate molecular weights of matrix peaks.

### DETAILED DESCRIPTION

Hereinafter, the present invention will be described in detail.

### Cosmetic Composition

The present invention provides a cosmetic composition including an effective substance.

The cosmetic composition may be in the form of, for example, a solution, gel, solid or paste anhydrous product, an emulsion obtained by dispersing an oil phase in an aqueous phase, a suspension, microemulsion, microcapsule, microgranule, or vesicular dispersions of ionic (liposome) or nonionic type. For example, the cosmetic composition may be provided in the form of a toner, milk lotion, cream, skin lotion, lotion, serum, essence, emulsion, powder, ointment, spray, gel, pack, cleanser, soap, shampoo, hair rinse, bath preparation, cleansing agent, or concealer stick. In addition, the cosmetic composition may be formulated in the form of a foam or an aerosol composition further containing compressed propellants.

In addition, the cosmetic composition may contain auxiliaries commonly used in the cosmetic field, such as fatty substances, organic solvents, solubilizers, thickening and gelling agents, emollients, antioxidants, suspending agents, stabilizers, foaming agents, fragrances, surfactants, water, ionic or nonionic emulsifiers, fillers, metal ion sequestrants and chelating agents, preservatives, vitamins, blockers, humectants, essential oils, dyes, pigments, hydrophilic or lipophilic active agents, lipid vesicles, or any other ingredients conventionally used in cosmetics, in addition to the effective substance of the present invention.

In the cosmetic composition containing the effective substance of the present invention, the effective substance of the present invention may be added in an amount of about 0.1% by weight to about 50% by weight, preferably about 1% by weight to about 10% by weight, based on the total weight of the cosmetic composition.

When the effective substance of the present invention is used as a topical preparation for skin, the cosmetic composition may further include auxiliaries commonly used in the field of skin science, such as fatty substances, organic solvents, solubilizers, thickening and gelling agents, emollients, antioxidants, suspending agents, stabilizers, foaming agents, fragrances, surfactants, water, ionic or nonionic emulsifiers, fillers, metal ion sequestrants and chelating agents, preservatives, vitamins, blockers, humectants, essential oils, dyes, pigments, hydrophilic or lipophilic active agents, lipid vesicles, or any other ingredients conventionally used in topical preparations for skin. Furthermore, these ingredients may be incorporated in amounts conventionally used in the field of skin science.

The following examples are provided to illustrate the present invention in further detail. These examples are for illustrative purposes only and should not be construed as limiting the scope of the invention.

### <Example 1> Preparation of Mixed-Enzyme-Treated and Low-Temperature-Aged Hyaluronic Acid Hydrolysate (Weight Ratio of Proteinase to Pectinase in Mixed Enzymes=3:1)

### Process 1: Mixed Enzyme Treatment

To 100 g of hyaluronic acid powder (200 mesh), 3 g of Flavourzyme (proteinase, manufactured by Novozyme) and 1 g of Pectinex Ultra SP-L (pectinase, manufactured by Novozyme) were added as mixed enzymes, followed by addition of 300 mL of water, and the mixture was allowed to react at 50° C. for 120 minutes.

### Process 2: Low-Temperature Aging Treatment

The mixed-enzyme-treated hyaluronic acid prepared in Process 1 was subjected to primary steaming at 80° C. for 3 hours, followed by low-temperature aging at 16° C. for 3 days.

The hyaluronic acid hydrolysate subjected to both enzyme treatment and low-temperature aging was extracted by stirring with 3 L of purified water at 80° C. for 120 minutes, followed by concentration under reduced pressure and freeze-drying to obtain a mixed-enzyme-treated and low-temperature-aged hyaluronic acid hydrolysate.

### <Example 2> Preparation of Mixed-Enzyme-Treated and Low-Temperature-Aged Hyaluronic Acid Hydrolysate (Weight Ratio of Proteinase to Pectinase in Mixed Enzymes=2:1)

A hyaluronic acid hydrolysate was obtained by following the same procedure as Example 1, except that in Process 1 of Example 1, proteinase and pectinase were used at a weight ratio of 2:1 from the total 4 g of mixed enzymes.

### <Example 3> Preparation of Mixed-Enzyme-Treated and Low-Temperature-Aged Hyaluronic Acid Hydrolysate (Weight Ratio of Proteinase to Pectinase in Mixed Enzymes=4:1)

A hyaluronic acid hydrolysate was obtained by following the same procedure as Example 1, except that in Process 1 of Example 1, proteinase and pectinase were used at a weight ratio of 4:1 from the total 4 g of mixed enzymes.

### <Example 4> Preparation of Mixed-Enzyme-Treated and Low-Temperature-Aged Hyaluronic Acid Hydrolysate (Weight Ratio of Proteinase to Pectinase in Mixed Enzymes=1:1)

A hyaluronic acid hydrolysate was obtained by following the same procedure as Example 1, except that in Process 1 of Example 1, proteinase and pectinase were used at a weight ratio of 1:1 from the total 4 g of mixed enzymes.

### <Example 5> Preparation of Mixed-Enzyme-Treated and Low-Temperature-Aged Hyaluronic Acid Hydrolysate (Weight Ratio of Proteinase to Pectinase in Mixed Enzymes=5:1)

A hyaluronic acid hydrolysate was obtained by following the same procedure as Example 1, except that in Process 1 of Example 1, proteinase and pectinase were used at a weight ratio of 5:1 from the total 4 g of mixed enzymes.

### <Comparative Example 1> Preparation of Hyaluronic Acid (Without Mixed Enzymes and Low-Temperature Aging Treatments)

Without performing Processes 1 and 2 of Example 1, hyaluronic acid was obtained by adding 3 L of purified water to 100 g of hyaluronic acid powder (200 mesh), extracting with stirring at 80° C. for 120 minutes, followed by concentration under reduced pressure and freeze-drying.

Specifically, hyaluronic acid was obtained by adding 3 L of purified water to 100 g of hyaluronic acid powder (200 mesh), extracting at 80° C. for 120 minutes, followed by concentration under reduced pressure and freeze-drying.

### <Comparative Example 2> Preparation of Mixed-Enzyme-Treated Hyaluronic Acid Hydrolysate (Without Low-Temperature Aging Treatment)

A mixed-enzyme-treated hyaluronic acid hydrolysate was prepared following the same procedure as Example 1, except that Process 2 was not performed.

Specifically, to 100 g of hyaluronic acid powder (200 mesh), 3 g of proteinase and 1 g of pectinase were added and mixed, followed by addition of 300 mL of water, and the mixture was allowed to react at 50° C. for 120 minutes. The mixed-enzyme-treated hyaluronic acid was extracted by stirring with 3 L of purified water at 80° C. for 120 minutes, followed by concentration under reduced pressure and freeze-drying to obtain a mixed-enzyme-treated hyaluronic acid hydrolysate.

### <Comparative Example 3> Preparation of Single-Enzyme-Treated Hyaluronic Acid Hydrolysate (With Single-Enzyme Treatment and Without Low-Temperature Aging Treatment)

A single-enzyme-treated hyaluronic acid hydrolysate was prepared following the same procedure as Example 1, except that 1 g of proteinase was used instead of the mixed enzymes in Process 1 and Process 2 was not performed.

Specifically, to 100 g of hyaluronic acid powder (200 mesh), 1 g of proteinase was added, followed by addition of 300 mL of water, and the mixture was allowed to react at 50° C. for 120 minutes. The single-enzyme-treated hyaluronic acid was extracted by stirring with 3 L of purified water at 80° C. for 120 minutes, followed by concentration under reduced pressure and freeze-drying to obtain a single-enzyme-treated hyaluronic acid hydrolysate.

### <Comparative Example 4> Preparation of Low-Temperature-Aged Hyaluronic Acid Hydrolysate (Without Mixed Enzyme Treatment)

A low-temperature-aged hyaluronic acid hydrolysate was prepared following the same procedure as Example 1, except that Process 1 was not performed.

Specifically, 100 g of hyaluronic acid powder (200 mesh) was subjected to primary steaming at 80° C. for 3 hours, followed by aging at 16° C. for 3 days. The low-temperature-aged hyaluronic acid was extracted by stirring with 3 L of purified water at 80° C. for 120 minutes, followed by concentration under reduced pressure and freeze-drying to obtain a low-temperature-aged hyaluronic acid hydrolysate.

### <Comparative Example 5> Preparation of Proteinase-Treated Hyaluronic Acid Hydrolysate (With Single-Enzyme and Low-Temperature Aging Treatments)

A hyaluronic acid hydrolysate was obtained by following the same procedure as in Example 1, except that 4 g of proteinase alone was used instead of the mixed enzymes.

### <Comparative Example 6> Preparation of Pectinase-Treated Hyaluronic Acid Hydrolysate (With Single Enzyme and Low-Temperature Aging Treatments)

A hyaluronic acid hydrolysate was obtained by following the same procedure as in Example 1, except that 4 g of pectinase alone was used instead of the mixed enzymes.

### <Comparative Example 7> Preparation of Mixed-Enzyme-Treated and Low-Temperature-Aged Hyaluronic Acid Hydrolysate (Weight Ratio of Proteinase to Pectinase in Mixed Enzymes=0.8:1)

A hyaluronic acid hydrolysate was obtained by following the same procedure as in Example 1, except that in Process 1, proteinase and pectinase were used at a weight ratio of 0.8:1 from the total 4 g of mixed enzymes.

### <Comparative Example 8> Preparation of Mixed-Enzyme-Treated and Low-Temperature-Aged Hyaluronic Acid Hydrolysate (Weight Ratio of Proteinase to Pectinase in Mixed Enzymes=5.2:1)

A hyaluronic acid hydrolysate was obtained by following the same procedure as in Example 1, except that in Process 1, proteinase and pectinase were used at a weight ratio of 5.2:1 from the total 4 g of mixed enzymes.

### <Experimental Example 1> Cytotoxicity Test: WST Assay

To confirm whether the hyaluronic acid samples prepared in Example 1 and Comparative Examples 1 to 4 cause cellular irritation, a cytotoxicity test was conducted using macrophages (RAW 264.7 cells) as the target.

RAW 264.7 cells, a type of macrophage, were equally counted and seeded at 1.0×10⁴ cells/well into a 24-well plate using DMEM medium containing 1% penicillin/streptomycin and 10% FBS (fetal bovine serum), followed by culture for 24 hours at 37° C. under 5% CO₂. After 24 hours of culture, the hyaluronic acid samples prepared in Example 1 and Comparative Examples 1 to 4 were mixed with the medium at various concentrations (0%, 0.5%, 1%, 2%, 4%), and 1 mL of each mixture was added to each well and allowed to react for 24 hours at 37° C. under 5% CO₂. Subsequently, after removing only the supernatant from each well, WST-1 assay solution (ez-cytox) was added to each well. After reacting for 2 hours in an incubator, the absorbance at 450 nm was measured using an ELISA reader.

Cell viability was calculated using Equation 1 by comparing with the groups untreated with the samples (untreated groups).

### Equation 1

Cell Viability (%)=(Absorbance of Sample-Treated Group/Absorbance of Untreated Group)×100

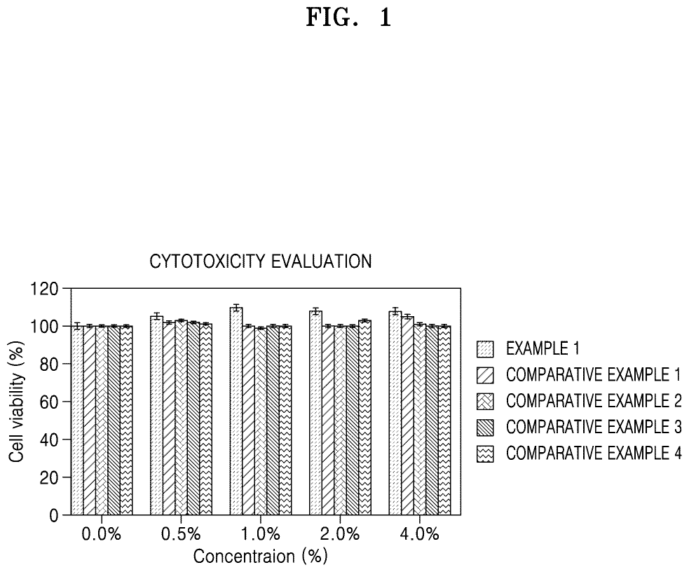

FIG. 1 is a graph showing results of cytotoxicity tests of the hyaluronic acid samples prepared in Example 1 and Comparative Examples 1 to 4.

As shown in FIG. 1, all hyaluronic acid samples prepared in Example 1 and Comparative Examples 1 to 4 showed no cytotoxicity, confirming their safety for use as cosmetic materials.

### <Experimental Example 2> Irritation Alleviation Test: Histamine Inhibition Activity

Beta-hexosaminidase (β-hexosaminidase) is a substance that constitutes the granules of mast cells, and histamine secretion due to mast cell degranulation is proportional to the amount of beta-hexosaminidase released. Therefore, the degranulation and histamine secretion inhibitory effects of the hyaluronic acid samples were confirmed by measuring the amount of beta-hexosaminidase released from RBL-2H3 cells, a rat mast cell line. After suspending RBL-2H3 cells in DMEM containing 10% FBS, 2×10⁵ cells per well were dispensed into a 24-well plate. The cells were then sensitized with 0.5 μg/mL DNP-IgE per well and incubated overnight in a 5% CO₂ incubator. Subsequently, the cells in each well were washed twice with Siraganian buffer (119 mM NaCl, 5 mM KCl, 5.6 mM glucose, 0.4 mM MgCl₂, 25 mM HEPES, 40 mM NaOH, 1 mM CaCl₂, 0.1% BSA, pH 7.2), pre-incubated with Siraganian buffer at 37° C. for 10 minutes, and then incubated for another 10 minutes after addition of the test substances. Subsequently, the cells were treated with antigen (DNP-BSA, 10 μg/mL) at 37° C. for 30 minutes to induce degranulation, followed by termination of the reaction on ice for 10 minutes. 20 μL of the supernatant was transferred to a 96-well plate, and 1 mM p-nitrophenyl-N-acetyl-β-D-glucosaminide was added followed by 1 hour incubation. After addition of stop solution (0.1 M Na₂CO₃/NaHCO₃), the absorbance was measured at 405 nm using an ELISA reader for quantification.

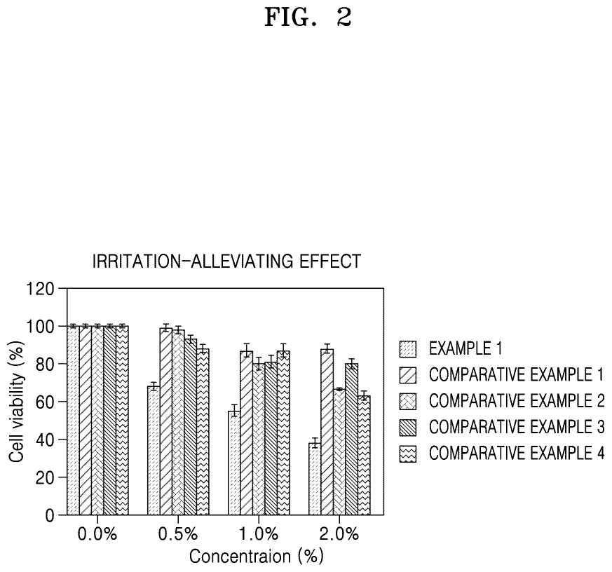

FIG. 2 is a graph showing results of histamine inhibition activity of the hyaluronic acid samples prepared in Example 1 and Comparative Examples 1 to 4.

As shown in FIG. 2, the hyaluronic acid sample prepared in Example 1 exhibited significantly superior histamine inhibition activity compared to Comparative Examples 1 to 4, wherein the histamine inhibition activity improved in a concentration-dependent manner up to a treatment concentration of 2.0%.

### <Experimental Example 3> Anti-inflammatory Efficacy Test: Nitric Oxide (NO) Production Inhibitory Activity

To measure the anti-inflammatory activity of the hyaluronic acid samples prepared in Example 1 and Comparative Examples 1 to 4, experiments were performed to measure the concentration of NO produced by inflammation-induced reactions.

RAW 264.7 cells, a type of macrophage, were equally counted and seeded at 1.0×10⁴ cells/well into a 24-well plate using DMEM medium containing 1% penicillin/streptomycin and 10% FBS, followed by culture for 24 hours at 37° C. under 5% CO₂. After 24 hours of culture, the hyaluronic acid samples prepared in Example 1 and Comparative Examples 1 to 4 were mixed with the medium at a concentration of 50 μg/mL. Each well was then supplemented with 1 mL of this mixture and allowed to react for 24 hours in an incubator at 37° C. and 5% CO₂. In addition, lipopolysaccharide (LPS), an inflammatory inducer that promotes NO expression, was also added at a concentration of 1 μg/mL and allowed to react for 24 hours at 37° C. under 5% CO₂. Subsequently, after removing only the supernatant from each well, 100 μL of the culture medium was transferred to a 96-well plate using a NO detection kit. 50 μL each of Griess reagent A (N-1-naphthylethylenediamine, NEDHC) and Griess reagent B (sulfanilamide) were added and allowed to react for 10 minutes. The absorbance was then measured at 540 nm using an ELISA plate reader.

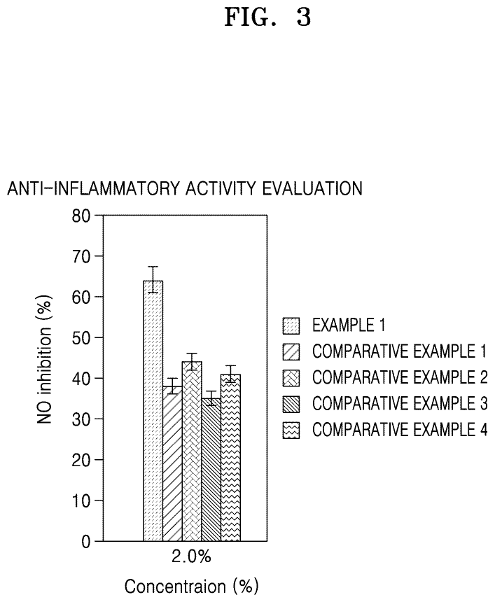

FIG. 3 is a graph showing results of nitric oxide (NO) production inhibitory activity of the hyaluronic acid samples prepared in Example 1 and Comparative Examples 1 to 4.

As shown in FIG. 3, the hyaluronic acid sample prepared in Example 1 exhibited significantly superior NO production inhibitory activity compared to Comparative Examples 1 to 4.

### <Experimental Example 4> Moisturization Enhancement Test: Clinical Evaluation of Skin Moisture Improvement up to 20 Layers

A clinical evaluation was conducted to measure the moisturizing enhancement effect of the hyaluronic acid samples prepared in Example 1 and Comparative Examples 1 to 4.

The hyaluronic acid samples prepared in Example 1 and Comparative Examples 1 to 4 were diluted to a concentration of 50 μg/mL and applied to the forearm area located 10 cm from both wrists of the subjects. The moisture on the skin surface was measured using an Epsilon E100, and the moisture content within the skin was measured by repeatedly stripping using Scotch Magic Invisible Tape (manufacturer: 3M) applied with the same pressure for 2 seconds.

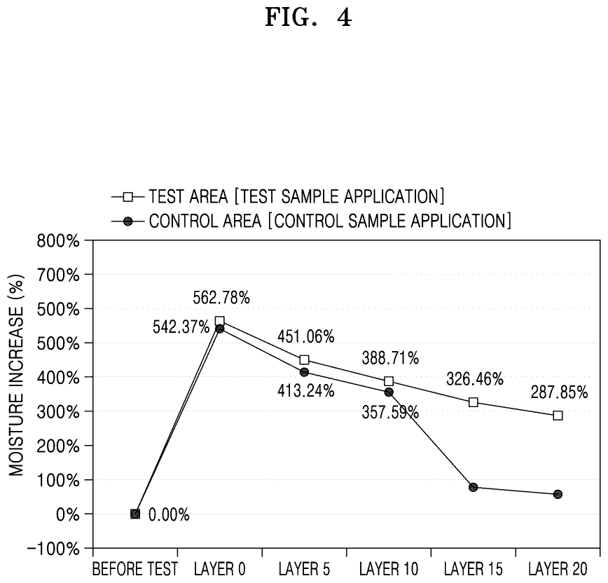

FIG. 4 is a graph showing the increase rate in skin moisture content according to treatment with the hyaluronic acid sample prepared in Example 1 versus a control sample (Comparative Example 1).

As shown in FIG. 4, the group treated with the low-molecular-weight hyaluronic acid sample prepared in Example 1 demonstrated superior moisturizing effects up to the 20th skin layer compared to the control sample (Comparative Example 1).

### <Experimental Example 5> Evaluation of Hyaluronic Acid Molecular Weight

The molecular weights of the hyaluronic acid hydrolysates obtained in Comparative Examples 1 to 8 and Examples 1 to 5 were measured and compared.

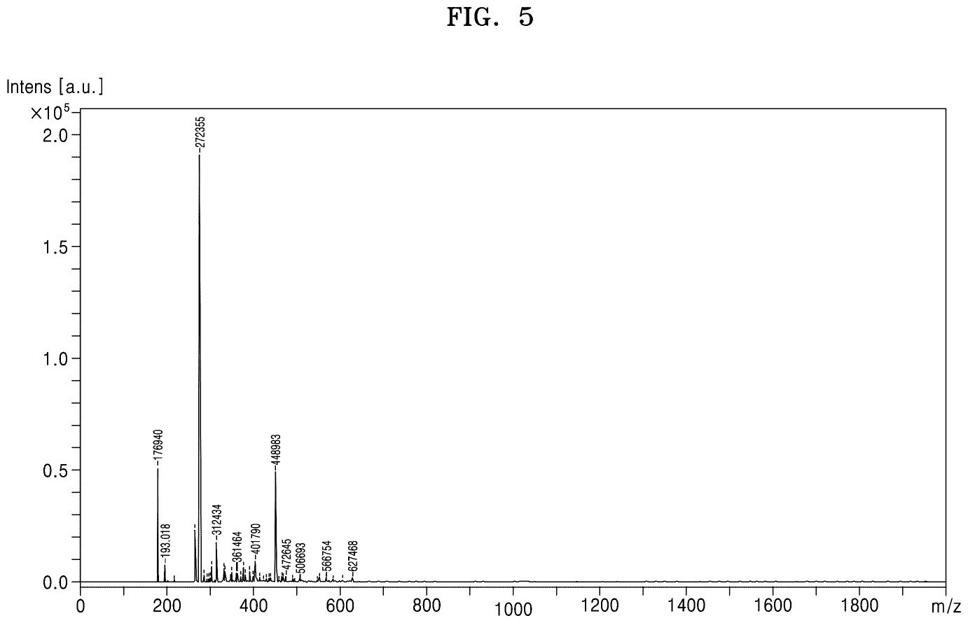

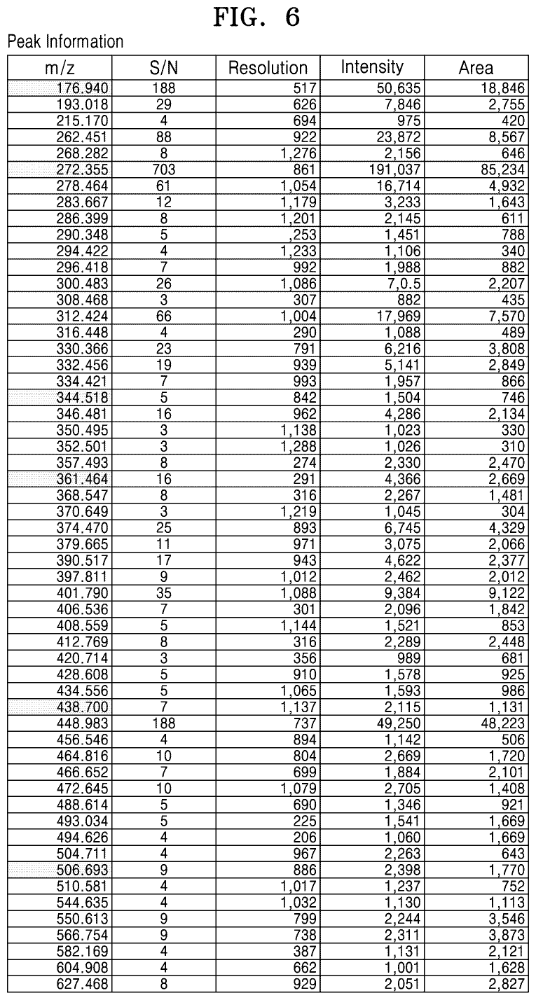

The molecular weight of the mixed-enzyme-treated and low-temperature-aged hyaluronic acid hydrolysate of Example 1 was analyzed for molecular weight confirmation at Gyeonggido Business & Science Accelerator (GBSA), and the results are shown in FIGS. 5 and 6.

For molecular weight measurement, a matrix solution was prepared by adding DHB (2,5-dihydroxybenzoic acid) at 10 mg/mL to a solvent of 0.1% TFA/ACN (1:1, v/v). 2 μL of the Example 1 sample was directly mixed with 2 μL of the matrix solution on a MALDI target and vacuum-dried. An autoflex maX (manufactured by Bruker Daltonics) was used as the molecular weight measurement instrument. The instrument parameters were as follows:
1. Instrument Control: Flex Control 3.4 (Bruker Daltonics)
2. Analysis Mode: Linear Mode
3. Polarity: Positive
4. Detection: m/z 100-100,000
5. Laser Repetition Rate: 2,000 Hz
6. Number of Shots: 500 shots
7. Deflection: On, 100 Da
8. Voltage

| Part | Voltage |
|---|---|
| Ion Source I | 19.00 kV |
| Ion Source II | 16.85 kV |
| Lens | 8.55 kV |

FIG. 5 is a spectrum image measuring the molecular weight of the hyaluronic acid hydrolysate of Example 1.

FIG. 6 is a table summarizing peak information from the spectrum measuring the molecular weight of the hyaluronic acid hydrolysate of Example 1. The gray cells indicate molecular weights of matrix peaks.

As shown in FIGS. 5 and 6, it was confirmed that the weight-average molecular weight excluding the matrix peaks is 368.3 Da.

Using the same molecular weight measurement method as in Example 1, the molecular weights of the hyaluronic acid obtained in Examples 2 to 5 and Comparative Examples 1-8 were measured, and the results are shown in Table 1 and Table 2 below, respectively.

TABLE 1

| | Mixed enzymes | | Low- | Weight- |
|---|---|---|---|---|
| | (total weight 4 g, | | temperature | average |
| | weight ratio) | | aging | molecular |
| | Proteinase | Pectinase | treatment | weight (Da) |
| Comparative | 0.8 | 1 | Treated | 3512.8 |
| Example 7 | | | | |
| Example 4 | 1 | 1 | Treated | 487.3 |
| Example 2 | 2 | 1 | Treated | 412.1 |
| Example 1 | 3 | 1 | Treated | 368.3 |
| Example 3 | 4 | 1 | Treated | 405.4 |
| Example 5 | 5 | 1 | Treated | 451.7 |
| Comparative | 5.2 | 1 | Treated | 3217.1 |
| Example 8 | | | | |

TABLE 2

| | Mixed enzymes | | Low- | Weight- |
|---|---|---|---|---|
| | (total weight 4 g, | | temperature | average |
| | weight ratio) | | aging | molecular |
| | Proteinase | Pectinase | treatment | weight (Da) |
| Comparative | 0 | 0 | Untreated | 1,547,269 |
| Example 1 | | | | |
| Comparative | 3 | 1 | Untreated | 12,014 |
| Example 2 | | | | |
| Comparative | 1 | 0 | Untreated | 150,328 |
| Example 3 | | | | |
| Comparative | 0 | 0 | Treated | 251,476 |
| Example 4 | | | | |
| Comparative | 4 | 0 | Treated | 13,516 |
| Example 5 | | | | |
| Comparative | 0 | 4 | Treated | 14,117 |
| Example 6 | | | | |

As shown in Table 1, it was confirmed that when the weight ratio of proteinase to pectinase in the mixed enzymes was used in the range of 1:1 to 5:1, the hydrolysis rate of hyaluronic acid was significantly higher.

As shown in Table 2, Comparative Examples 1 and 4 are samples that differ only in whether low-temperature aging treatment was applied. Comparing these samples confirmed that low-temperature aging treatment has a hydrolysis effect. This result can also be confirmed by comparing Comparative Example 2 with Example 1. Additionally, Comparative Examples 5 and 6 are samples treated with a single enzyme and are compared with Examples 1 to 5. Comparing these samples confirmed that the hydrolysis rate was significantly improved in the mixed-enzyme-treated samples compared to the single-enzyme-treated samples.”

### Preparation Examples of Cosmetic Preparations

The effective substance according to the present invention may be formulated into various types of cosmetic preparations depending on the purpose. The following are examples of methods for preparing some cosmetic preparations containing the effective ingredient of the present invention as an active ingredient, but the present invention is not limited thereto.

### <Cosmetic Preparation Example 1> Preparation of Softening Skin Toner

| | |
|---|---|
| Effective Substance | 10.0 parts by weight |
| 1,3-Butylene Glycol | 1.00 part by weight |
| Disodium EDTA | 0.05 parts by weight |
| Allantoin | 0.10 parts by weight |
| Dipotassium Glycyrrhizate | 0.05 parts by weight |
| Citric Acid | 0.01 parts by weight |
| Sodium Citrate | 0.02 parts by weight |
| Glycereth-26 | 1.00 part by weight |
| Arbutin | 2.00 parts by weight |
| Hydrogenated Castor Oil | 1.00 part by weight |
| Ethanol | 30.0 parts by weight |
| Preservative | Trace amount |
| Colorant | Trace amount |
| Fragrance | Trace amount |
| Purified Water | Balance |

### <Cosmetic Preparation Example 2> Preparation of Nutritive Cream

| | |
|---|---|
| Effective Substance | 10.0 parts by weight |
| 1,3-Butylene Glycol | 7.00 parts by weight |
| Glycerin | 1.00 part by weight |
| D-Panthenol | 0.10 parts by weight |
| Plant Extract | 3.20 parts by weight |
| Magnesium Aluminum Silicate | 0.30 parts by weight |
| PEG-40 Stearate | 1.20 parts by weight |
| Stearic Acid | 2.00 parts by weight |
| Polysorbate 60 | 1.50 parts by weight |
| Lipophilic Glyceryl Stearate | 2.00 parts by weight |
| Sorbitan Sesquioleate | 1.50 parts by weight |
| Cetearyl Alcohol | 3.00 parts by weight |
| Mineral Oil | 4.00 parts by weight |
| Squalane | 3.80 parts by weight |
| Caprylic/Capric Triglyceride | 2.80 parts by weight |
| Vegetable oil | 1.80 parts by weight |
| Dimethicone | 0.40 parts by weight |
| Dipotassium Glycyrrhizate | Trace amount |
| Allantoin | Trace amount |
| Sodium Hyaluronate | Trace amount |
| Tocopheryl Acetate | Appropriate amount |
| Triethanolamine | Appropriate amount |
| Preservative | Appropriate amount |
| Fragrance | Appropriate amount |
| Purified Water | Balance |

The low-molecular-weight hyaluronic acid prepared by the method according to the present invention achieves a weight-average molecular weight (Mw) of 500 Da or less, resulting in significantly improved skin absorption rate and can thus provide significantly superior effects in alleviating skin irritation and providing moisturization.

The foregoing description of various embodiments of the invention has been presented for purposes of illustration. It will be apparent to those skilled in the art that the present invention may be embodied in several modified forms without departing from the spirit of essential characteristics thereof. The embodiments disclosed herein should be therefore considered illustrative and not restrictive. The scope of the invention is defined by the appended claims rather than by the description preceding them, and all changes that fall within the metes and bounds of the claims, or equivalence of such metes and bounds thereof are therefore intended to be embraced by the claims.

What is claimed is:

1. A method of preparing low-molecular-weight hyaluronic acid, the method comprising:
mixing hyaluronic acid and mixed enzymes comprising proteinase and pectinase in distilled water, and conducting an enzymatic reaction to obtain mixed-enzyme-treated hyaluronic acid (Process 1); and
subjecting the mixed-enzyme-treated hyaluronic acid obtained from Process 1 to steam treatment, and subsequently to low-temperature aging treatment at 5° C. to 20° C. for 1 day to 5 days (Process 2).

2. The method of claim 1, wherein the proteinase in Process 1 is selected from the group consisting of Flavourzyme, Protamex, α-chymotrypsin, subtilisin Carlsberg, thermolysin, papain, fungal protease, and pepsin.

3. The method of claim 1, wherein the pectinase in Process 1 is selected from the group consisting of Pectinex Ultra SP-L, Viscozyme L, Ultrazyme AFP, Pectinex Ultra AFP, Pectinex Ultra Clear, and α-herbzyme.

4. The method of claim 1, wherein the mixed enzymes comprise 1 part by weight to 5 parts by weight of proteinase with respect to 1 part by weight of pectinase.

5. The method of claim 4, wherein the mixed enzymes comprise 2 parts by weight to 4 parts by weight of proteinase with respect to 1 part by weight of pectinase.

6. The method of claim 1, wherein, in Process 2, the steam treatment is conducted at 70° C. to 90° C. for about 2 hours to 4 hours, and the low-temperature aging treatment is conducted at 14° C. to 18° C. for 2 days to 4 days.

7. The method of claim 1, wherein the hyaluronic acid prepared by the method has a weight-average molecular weight (Mw) of 500 Da or less.

8. A low-molecular-weight hyaluronic acid prepared by the method of claim 1, having a weight-average molecular weight (Mw) of 500 Da or less.

9. A cosmetic composition for alleviating skin irritation and providing moisturization, comprising the low-molecular-weight hyaluronic acid prepared by the method of claim 1.

10. A cosmetic composition for alleviating skin irritation and providing moisturization, comprising a low-molecular-weight hyaluronic acid having a weight-average molecular weight (Mw) of 500 Da or less.
